(19) United States
(12) Patent Application Publication
(10) Pub. No.: US 2025/0345620 A1
(43) Pub. Date: Nov. 13, 2025

(54) NONINVASIVE IMAGE-GUIDED MAGNETOHYDRODYNAMIC FLUID MANIPULATION

(71) Applicant: Weinberg Medical Physics, Inc., North Bethesda, MD (US)

(72) Inventor: Irving N. WEINBERG, Chevy Chase, MD (US)

(21) Appl. No.: 19/202,133

(22) Filed: May 8, 2025

Related U.S. Application Data
(60) Provisional application No. 63/644,281, filed on May 8, 2024.

(51) Int. Cl. A61N 2/00; A61N 2/02

(52) U.S. Cl. CPC A61N 2/004; A61N 2/02

(57) ABSTRACT

An apparatus and method are provided for treating conditions affecting animal or human subjects. The apparatus and method treat conditions related to abnormal fluid transport, in which treatment is implemented by applying static and dynamic electromagnetic fields to exert pressure on the fluids.

### CROSS-REFERENCE AND PRIORITY CLAIM

This application claims priority to U.S. Provisional Patent Application Ser. No. 63/644,281, entitled “Noninvasive Image-Guided Magnetohydrodynamic Fluid Manipulation” filed May 8, 2024, the entirety of which is incorporated by reference.

### FIELD

An apparatus and method of treating conditions related to the enhancement of fluid transport in the brain are provided. In particular, an apparatus and method of applying static and dynamic electromagnetic fields for treating conditions related to abnormal fluid flow are provided.

### BACKGROUND

Magnetohydrodynamics (MHD) can be used to move conductive fluids and to move materials that are transported by the conductive fluids. MHD describes forces on moving electrical charges in the presence of magnetic fields. The magnitude of this force is J X B, where X is a cross product, J is the current vector represented by the moving charges, and B is the applied magnetic field vector. A description of the MHD force on conductive fluid in the inner ear when a patient is placed in a high-field (4 Tesla) magnetic resonance imaging (MRI) system was provided by J.F. Schenck in the 1992 Annals of the New York Academy of Science paper entitled “Health and Physiological Effects of Human Exposure to Whole-Body Four-Tesla Magnetic Fields during MRI”. Schenck suggested that when a subject would move his or her head, the MHD force from the MRI's static magnetic field would lead to pressure on the ear canal fluid, and that this pressure might be noticeable by the subject.

### SUMMARY

An apparatus and method are provided for treating conditions affecting animal or human subjects. The apparatus and method treat conditions related to abnormal fluid transport, in which treatment is implemented by applying static and dynamic electromagnetic fields to exert pressure on the fluids.

### BRIEF DESCRIPTION OF THE FIGURES

FIG. 1 shows an embodiment of the apparatus acting on a body part.

FIG. 2 shows a flowchart a treatment method according to the disclosed embodiments.

### DETAILED DESCRIPTION

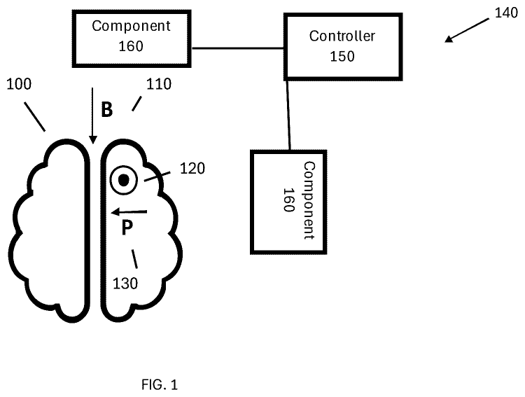

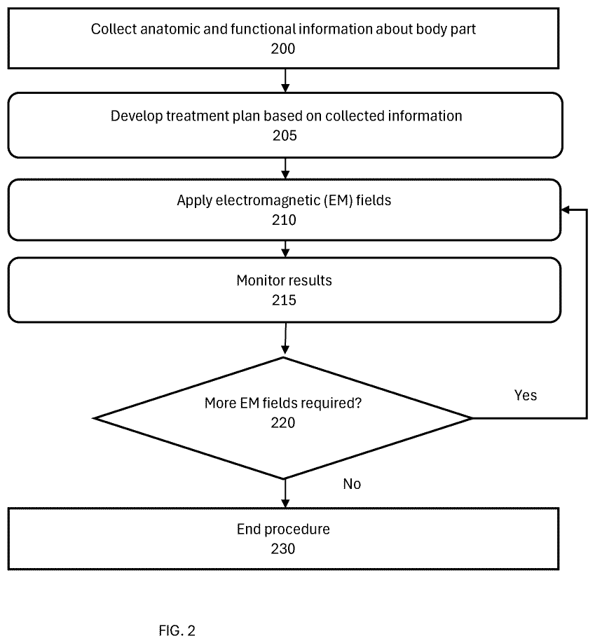

Disclosed embodiments are described in relation to an apparatus illustrated in FIG. 1 and a method in FIG. 2 for treating animal or human subjects that have conditions related to abnormal fluid transport in a body part or other illnesses or other conditions requiring flow enhancement. An example of such an illness would be normal pressure hydrocephalus, in which the normal flow of cerebrospinal fluid is blocked, causing enlargement of the brain's ventricles. An example of another such illness would be misfolded protein in the brain, as can be seen in Alzheimer disease.

Referring to FIG. 1, a body part 100 (illustrated as a brain) is exposed to a magnetic field 110. In an embodiment, magnetic field, represented by magnetic field vector, 110, is generated by an electropermanent magnet system 140, said system containing components 160 capable of collecting images as well as generating magnetic fields, as disclosed in U.S. Pat. No. 10,908,240, entitled “METHOD FOR ACQUIRING AN IMAGE AND MANIPULATING OBJECTS WITH MAGNETIC GRADIENTS PRODUCED BY ONE OR MORE ELECTROPERMANENT MAGNET”. An advantage of using the electropermanent magnets in the disclosed embodiments is that the orientation and magnitude of the magnetic vector fields generated by such electropermanent magnets may be changed electronically (rather than mechanically), and so for the purposes of this disclosure, said magnetic fields are denoted as quasi-static magnetic fields, meaning that they once the magnetic field has been established (e.g., by running a current through a coil through the electropermanent magnet), the magnetic field may continue indefinitely until changed (for example by applying another current to the electropermanent magnet).

As seen in FIG. 1, another electromagnetic field vector 120 (e.g., an electric field capable of generating current in the vector's direction) is applied to the body part. This may be a transient electric field generated by induction from coils outside the body part, or by applying current through electrodes attached to the body part, or through some other mechanism. The vector direction of the vector 120 is not parallel to the direction of magnetic field vector 110. The direction of vector 120 is shown as an arrow pointing out of the plane of the image. The pressure created by the MHD force is shown as vector P (130), which may cause conducting fluid to move in the direction of P or have other beneficial effects. For example, the pressure may change the conformation of proteins.

It is understood that electromagnetic field vector 120 may generate currents in electrically conductive fluid components (e.g., cerebrospinal fluid) within the body part 100, and MHD may exert a pressure 130 on these fluid components. Said pressure may transport the fluid components to and from locations in the body part as needed to treat the condition (for example, normal pressure hydrocephalus or sleep deficiency). Said pressure may unfold misfolded proteins, or may transport pathological substances (for example, abnormally accumulated increased waste products of brain metabolism) from the body part. Said abnormal accumulation may be due to lack of sufficient sleep.

In some embodiments, the system 140 may generate magnetic field 110 or electromagnetic field 120 by permanent magnets or superconducting magnets or radiofrequency antennas whose position or orientation or magnitude may be adjusted in time or space. A conventional MRI generally only applies a static field in one direction, and with one magnitude. It is understood that the system may further have a controller 150 such as a processor or computer, graphical user interface, connectors, cables, mounting devices, and power supplies.

For the purposes of this disclosure, the terms magnetic and electromagnetic are used interchangeably, since it is understood that every changing magnetic field has an electrical component and vice versa.

It is understood that particles such as iron-containing blood cells may be transported along with the conductive fluid components, and that the magnetic properties of such particles may be incorporated into a treatment plan.

Although the vectors 110 and 120 are shown for clarity of illustration as being of specific directions, it is understood that these fields may have different directions, and that the field properties may vary in space, and may include multiple magnetic fields (and not just two as shown).

It is understood that a current in the direction of vector 120 may be generated in the body part through means other than magnets, for example by applying electrodes to the surface of the body part, or to surfaces near the body part so that current flows in the body part.

For the purposes of this disclosure, the means of establishing the quasi-static field 110 and the electromagnetic vector 120 (said vector 120 capable of generating currents in the body part) are described as components with directions adjustable by the operator (that is, in generalized directions), meaning (for the purposes of this disclosure) that the direction and currents of these fields can be adjusted at least in part (for example by more than 15-degrees) electronically or mechanically without moving the body part.

Referring to FIG. 2, a procedure is disclosed for treating a condition. Information is collected in operation 200 to guide the development of a treatment plan. This information may be collected using the functionality of the electropermanent MRI described in invention 10908240. The information may include anatomic data (for example, the location and size of the brain ventricles) and/or functional data (for example, the flow of fluid using MRI pulse sequences sensitive to such flow). In some embodiments, some or all of the information is collected at a time when the procedure is contemplated. In some alternative embodiments, some or all of the information is collected at a time significantly earlier (for example, several months) than the procedure is contemplated. In operation 205, at least one computer is used to determine a treatment plan. The treatment plan may include a calculation of appropriate time, magnitude, and directions of the applied electromagnetic fields, taking into account the anatomic and/or functional information collected in operation 200. In operation 210, the electromagnetic fields are applied to the body part, in accordance with the treatment plan. For example, a quasi-static magnetic field 110 and transient magnetic gradients 120 may be applied, to generate a pressure P 130 acting on fluid in the body part. In some embodiments, the results of said application are monitored 215, for example by using MRI flow-sensitive pulse sequences to observe fluid flow in the body part. In some embodiments, a determination may be made 220, using monitoring or other information such as comparing the treatment plan in 205 with the monitored result in operation 215, (with the aid of a computer) whether to continue, repeat, modify or end the application of electromagnetic fields. If the decision has been made to end the procedure, due to satisfactory results, or conclusion of the treatment plan, then the procedure is ended 230.

In some embodiments, flow transport is augmented to remove pathological accumulations of fluid or of substances that may be transported by said fluid by the pressure application. In some embodiments, transport is augmented to remove pathological accumulations of substances in subjects with sleep deficits by the pressure application.

As an example of operation of an embodiment, a quasi-static magnetic field 110 with a magnitude of 100 milliTeslas is generated by an array of electropermanent magnets in the direction shown in FIG. 1. A transient magnetic gradient 120 is then applied to create an electric field magnitude of 100 volts per meter in the body part (said field being magnitude being typical of transcranial magnetic stimulation systems). If the apparatus is used to create electric fields deeper in the brain than typical transcranial magnetic fields, then the electric field magnitude may be smaller (for example, 100 volts per meter). It is understood that the same electropermanent magnets may be used to create magnetic fields 110 and 120. Assuming that the cerebrospinal fluid has a conductivity of about 1.5 siemens per meter, a transient current is established in the direction of vector 120 with a magnitude of 150 amperes. Then the force on the material that is carrying the current (for example the cerebrospinal fluid) may be about 10 newtons over the area of application (about 0.01 square meters), or 10,000 pascals. Said applied pressure may be substantially higher as compared to osmotic forces on cerebrospinal fluids (about 1,000 pascals). To maintain this applied pressure, the transient magnetic fields 120 may be applied repetitively, for example, between 10-100 times per second, 100-1,000 times per second, between 1,000-10,000 times per second, or between 100-10,000-1 million times per second.

In accordance with this disclosure, it should be understood that the action of the components of the system or apparatus is under the control of a computer. More specifically, it should be understood that the operations explained herein may be implemented in conjunction with, or under the control of, one or more general purpose computers running software algorithms to provide the presently disclosed functionality and turning those computers into specific purpose computers.

Moreover, those skilled in the art will recognize, upon consideration of the above teachings, that the above exemplary embodiments may be based upon use of one or more control devices such as programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out the above-described method operations and resulting functionality. In this case, the term non-transitory is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While certain illustrative embodiments have been described, it is evident that many alternatives, modifications, permutations and variations will become apparent to those skilled in the art in light of the foregoing description. Accordingly, the various embodiments of, as set forth above, are intended to be illustrative, not limiting. Various changes may be made without departing from the spirit and scope of the invention.

1. An apparatus for treating a body part within an animal or human with a condition of impaired flow within the body part, the apparatus comprising:
components for generating a quasi-static magnetic field of generalized direction applied to a body part within a human or non-human animal, and
second components for generating electrical currents of generalized direction within the body part, wherein the direction of the electrical currents is not parallel to the direction of the quasi-magnetic field, and
a control configured to control the components and second components so that a magnetohydrodynamic force of the quasi-static magnetic field upon the electrical current exerts a pressure on a fluid within the body part.

2. The apparatus of claim 1, wherein the components for generating a quasi-static magnetic field or the second components for generating electrical currents include at least one electropermanent magnet.

3. The apparatus of claim 1, wherein the components for generating a quasi-static magnetic field or the second components for generating electrical currents include at least one permanent magnet.

4. The apparatus of claim 1, wherein the components for generating a quasi-static magnetic field or the second components for generating electrical currents include at least one superconducting magnet.

5. The apparatus of claim 1, wherein the second components for generating currents include at least one coil.

6. The apparatus of claim 1, wherein the controller applies the transient magnetic field between ten and one million times per second.

7. A method for treating a body part within an animal or human with a condition of impaired flow within the body part, the method comprising:
generating a quasi-static magnetic field and applying it to the body part,
generating electric currents and applying them to the body part, and
controlling the generated quasi-static magnetic field and electric currents so that magnetohydrodynamic force of the quasi-static magnetic field upon the electrical currents exerts a pressure on a fluid within the body part that affects transport of the fluid or of body components affected by said transport.

8. The method of claim 7, wherein the pressure compensates for impaired flow.

9. The method of claim 7, further comprising collecting an image of the body part to form a treatment plan.

10. The method of claim 7, further comprising monitoring the quasi-static magnetic field and the electrical currents, or their effects during or immediately after therapeutic pressure application.

11. The method of claim 7, further comprising adjusting the quasi-static magnetic field or the electrical currents during therapeutic pressure application.

12. The method of claim 7, further comprising adjusting the quasi-static magnetic field or the electrical currents during therapeutic pressure application.

13. The method of claim 7, wherein flow transport is augmented to remove pathological accumulations of fluid or of substances that may be transported by said fluid.

14. The method of claim 7, wherein flow transport is augmented to remove pathological accumulations of substances in subjects with sleep deficits.
